(19) United States
(12) Patent Application Publication
(10) Pub. No.: US 2010/0255607 A1
(43) Pub. Date: Oct. 7, 2010

(54) AFFINITY CONTROL OF TAGGING NOISE

(76) Inventor: Roger W. Giese, Quincy, MA (US)

Correspondence Address: WEINGARTEN, SCHURGIN, GAGNEBIN & LEBOVICI LLP, TEN POST OFFICE SQUARE, BOSTON, MA, 02109, US

(21) Appl. No.: 12/743,462

(22) PCT Filed: Nov. 21, 2008
(86) PCT No.: PCT/US08/12978

§ 371 Date: May 18, 2010

Related U.S. Application Data
(60) Provisional application No. 61/003,895, filed on Nov. 21, 2007.

(51) Int. Cl. G01N 33/53

(52) U.S. Cl. 436/501

(57) ABSTRACT

Analytical methodology is disclosed in which a trace analyte in a sample is tagged to increase its signal strength for measurement, and an affinity substance is used at least after the tagging step (late in the method) to separate the tagged analyte from tagging noise, wherein the affinity substance is directed at the reactive part of the tag, at the reagent-reacted part of the tag, or at a nonreactive part of the analyte. This method includes the case where exposure of the sample to the affinity substance begins prior to and continues after the tagging reaction. The affinity substance comprises an affinity reagent such as an immobilized anti-body (immuno-affinity chromatography). This late use of an affinity substance (after the tagging reaction) is particularly important for methods in which trace analytes are reacted with a charge tag (tag possessing a charge) prior to detection by mass spectrometry.

### CROSS REFERENCE TO RELATED APPLICATIONS

This application claims the priority of U.S. Provisional Application No. 61/003,895 filed Nov. 21, 2007 entitled, AFFINITY CONTROL OF TAGGING NOISE, the whole of which is hereby incorporated by reference herein.

### STATEMENT REGARDING FEDERALLY SPONSORED RESEARCH OR DEVELOPMENT

N/A

### BACKGROUND OF THE INVENTION

In the field of analytical chemistry, one of the challenges is to set up analytical methods that provide enough sensitivity for an analyte of interest. Trace analytes are those that are present in a tiny amount (less than about one picomole) in a sample to be analyzed. This is especially true for some analytes in small biological samples such as samples of human tissue or blood. The most challenging case is a trace analyte that is present at a low concentration in a limited sample, since then the amount of the analyte available for detection is exceedingly small. Increasingly, such trace analytes need to be measured.

In principle, one can overcome the problem of trace analyte detection by derivatizing the analyte with a molecular tag having a signal group. A signal group is a molecular structure that is very sensitive in a detection method of interest. For a given trace analyte, a signal group is selected to give a tag-analyte product that has a much higher signal strength in the detection method of interest than the analyte. However, sensitivity is the ratio of signal-to-noise, and the performance of such tag-attachment assays is inevitably compromised for trace analytes by background signals in the detection method (tagging noise) from residual tag, tag byproducts (formed by reaction of tag with nonanalyte species, including solvent, in the tagging reaction), and contaminants in the tagging reagent. This problem tends to be severe because a large excess of the tag relative to the analyte is needed to efficiently label a trace analyte. Nonspecific forms of partitioning, chromatography, electrophoresis and other separation techniques can be used in an effort to remove the tagging noise. However, the effectiveness of these techniques can be limited because they may need to be used extensively to fully remove the diverse tagging noise towards a goal of maximizing sensitivity. Not only are such extensive removal procedures tedious, but they also tend to introduce new noise and cause losses of the tagged-analyte, thus canceling or limiting the gain in sensitivity from tagging the analyte.

Attempts have been made to overcome tagging noise after a tagging reaction by adding an “accessory reagent” to react with residual tag, yielding a tag byproduct (tag-accessory reagent) which is intended to be easier to remove from tag-analyte than ordinary tag byproducts. However, this technique has failed mainly because a relatively high amount of the accessory reagent is needed, which adds noise from residual accessory agent and its contaminants. Basically this just replaces one set of chemical noise with another, so that, once again, little or no gain in sensitivity tends to be achieved. Thus, apparently there is no ongoing practice of this technique for trace analytes.

As a consequence of persistent tagging noise, there is always a large a gap (e.g., 100 to 1000 fold) between the amount of tag-analyte that one can detect as a diluted, purified standard (prepared from a large amount of standard analyte) versus the amount of analyte that one can detect by actually subjecting a trace amount of it to a tagging reaction. However, only the latter option is available for trace analytes in real samples. This problem has held back the usefulness of derivatization with tagging reagents to enhance sensitivity for trace analytes throughout the history of analytical chemistry. Only for analytes at higher concentrations, above the trace level, is tagging sometimes a practical and effective technique to increase sensitivity. But this then, would not be trace analysis.

Affinity substances that recognize (mutual molecular fit like a lock and key) and thereby engage an analyte specifically have been used at the early stage of tagging in chemical analysis (prior to the tagging reaction) to extract an analyte from an initial impure sample (such as a biological sample) prior to derivatization (Morinello, E. J., Ham. A-J. L., Ranasinghe, A., Sangaiah, R., Swenberg, J. A., 2001. Simultaneous Quantitation of N²,3-Ethenoguanine and 1,N²-Ethenoguanine with an Immunoaffinity/Gas Chromatography/High-Resolution Mass Spectrometry Assay, Chem. Res. Toxicol., 14, 327-334). Affinity substances directed at a nonreactive part of the tag have been used to broadly isolate whatever is labeled by a given tag in a tagging reaction (Gygi, S. P., Aebersold, R., 2000. Mass spectrometry and proteomics 4, 489-494). Neither of these affinity strategies, alone or in combination, solves the long-standing problem of limited sensitivity due to tagging noise in trace analysis.

Affinity substances can be used in both immobilized and free forms. In an immobilized form, the affinity substance (such as an antibody) is attached covalently or noncovalently to a solid surface. This surface can be provided by an insoluble particle such as a chromatographic particle, a membrane, a filter or a relatively plain surface (which may be roughened or semi-porous) such as the bottom of well in a microtiter plate or the inside of a tube. The chromatographic particle may be porous or nonporous. In a free form, by definition, the affinity substance is simply dissolved in a solution. For example, in affinity filtration, a large affinity substance (such as an antibody) is used in a free form. After the free form of the affinity substance complexes the substance of interest, the complex is retained on an ultrafiltration membrane, and noncomplexed substances, if they are small enough, are removed by passage through the membrane. Either a free or immobilized form of an affinity substance can be used in affinity electrophoresis and in affinity centrifugation.

Organic groups having a permanent charge are an important class of signal groups for tags. Examples are quaternary amines and sulfonates. Analytes derivatized with charged tags are measured by mass spectrometry, an advantageous detection technique since it can provide high resolution and employ stable isotope internal standards. The use of charged tags to enhance detection of peptides by mass spectrometry has been reviewed (Keough, T., Youngquist, R. S., Lacey, M. P., 2003. Sulfonic Acid Derivatives for Peptide Sequencing by MALDI MS, Anal. Chem. 75, 156A-165A). Other examples of analytes that have been charge-tagged to improve their signal strength for detection by mass spectrometry are fatty acids (Yang, W-C., Adamec, J., Regnier, F. E., 2007. Enhancement of the LC/MS Analysis of Fatty Acids through Derivatization and Stable Isotope Coding, Anal. Chem. 79, 5150-5157), sugars (Naven, T. J. P., Harvey, D. J., 1996. Cationic Derivatization of Oligosaccharides with Girard's T Reagent for Improved Performance in Matrix-assisted Laser Desorption/Ionization and Electrospray Mass Spectrometry, Rapid Commun. Mass Spectrom. 10, 829-834), and a nucleoside (Hong, H., Wang, Y., 2007. Derivatization with Girard Reagent T Combined with LC-MS/MS for the Sensitive Detection of 5-Formyl-2′-deoxyuridine in Cellular DNA, Anal. Chem. 79, 322-326). However, in all of these and other reported tagging cases, high sensitivity is achieved only for the detection of diluted standards of the tagged analyte. For example, Hong and Wang report in the latter article that “[i]t is worth noting that the detection limit of FodU in the digested DNA sample was much poorer than that of the pure standard.” The barrier of tagging noise limits the performance of these methods for real samples.

### BRIEF SUMMARY OF THE INVENTION

Analytical methodology is disclosed in which a trace analyte in a sample is tagged to increase its signal strength for measurement, and an affinity substance is used at least after the tagging step (late in the method) to separate the tagged analyte from tagging noise, wherein the affinity substance is directed at the reactive part of the tag, at the reagent-reacted part of the tag, or at a nonreactive part of the analyte. This method includes the case where exposure of the sample to the affinity substance begins prior to and continues after the tagging reaction. The affinity substance comprises an affinity reagent such as an immobilized antibody (immuno-affinity chromatography). It is considered that this late use of an affinity substance (after the tagging reaction) is particularly important for methods in which trace analytes are reacted with a charge tag (tag possessing a charge) prior to detection by mass spectrometry. This is because the charged analyte tends to have the same charge as the background chemicals that produce the tagging noise, making the removal of this noise from the charged analyte particularly difficult. An affinity substance directed at the tagging reagent has the special advantage that the same affinity reagent can be applied to many analytes.

Thus, in general, the invention is directed to an assay for detecting the amount of a trace analyte AB in a sample that includes the steps of (1) providing an assay mixture including a sample in which an amount of a trace analyte AB is to be determined; (2) contacting the analyte AB in the assay mixture with a tagging reagent XYZ, to give the covalent product AB-YX along with residual XYZ and/or byproducts XY′, wherein B is the part of analyte AB that reacts with XYZ, A is the nonreactive part of AB, X is a signal group in XYZ, YZ is a reactivity group in XYZ, Z is the part of the reactivity group YZ that is replaced by AB, and XY′ is any product that forms when XYZ reacts with a reagent including solvent; and (3) measuring the amount of AB-YX in a free form from the assay mixture, wherein the amount of AB-YX from the assay mixture gives the amount of trace analyte AB in the sample.

The assay according to the invention includes the improvement of contacting the assay mixture with at least one affinity substance at least after the step of contacting the analyte AB in the assay mixture with a tagging reagent XYZ, wherein said at least one affinity substance is directed at the YZ part of XYZ, the Y′ part of XY′ or the A part of AB-YX. The assay according to the invention includes the condition wherein the assay mixture is contacted with the affinity substance before the step of contacting the analyte AB in the assay mixture with the tagging reagent XYZ and the affinity substance remains in the assay mixture during the step of contacting the analyte AB in the assay mixture with the tagging reagent XYZ. The A part of AB may be rendered nonreactive by complexing it with an affinity reagent prior to the tagging reaction.

Preferably, in the assay according to the invention, AB-XY is detected by fluorescence or by mass spectrometry. Preferably, at least one affinity substance is a solid phase reagent and is selected from the group consisting of immobilized or free forms of proteins, peptides, antibodies, antibody fragments, aptmers and molecular-imprinted polymers. X preferably is a charged organic group or an electrophoric group. Alternatively, X is preferably a fluorophore, lumiphore, electrochemically-active group, or radionucleotide. AB is preferably a steroid, lipid, carbohydrate, peptide, vitamin, drug or DNA adduct.

### BRIEF DESCRIPTION OF THE SEVERAL VIEWS OF THE DRAWINGS

Other features and advantages of the invention will be apparent from the following description of the preferred embodiments thereof and from the claims, taken in conjunction with the accompanying drawings, in which:

FIG. 1 shows tag detection of trace analyte AB facilitated by post-tagging affinity chromatography directed at the reactive end of the tag according to the method of the invention;

FIG. 2 shows tag detection of trace analyte AB facilitated by post-tagging affinity chromatography directed at the end of the tag that has undergone a side reaction according to the method of the invention;

FIG. 3 shows tag detection of trace analyte AB facilitated by affinity chromatography directed at the A end of the analyte after the tagging reaction according to the method of the invention;

FIG. 4 shows tag detection of analyte AB facilitated by use of an affinity substance directed at the analyte both during and after the tagging reaction according to the method of the invention;

FIG. 5 shows tag detection of a DNA adduct (analyte AB) facilitated by post-tagging use of two affinity substances according to the method of the invention;

FIG. 6 shows tag detection of 4-aminobiphenyl (analyte AB) facilitated by a post-tagging affinity step according to the method of the invention;

FIG. 7 shows tag detection of a peptide in a biosample facilitated by use of an affinity substance both during and after the tagging reaction according to the method of the invention; and

FIG. 8 shows tag detection of a DNA adduct facilitated by use of the same immobilized antibody as an affinity reagent both before and after a tagging reaction according to the method of the invention.

### DETAILED DESCRIPTION OF THE INVENTION

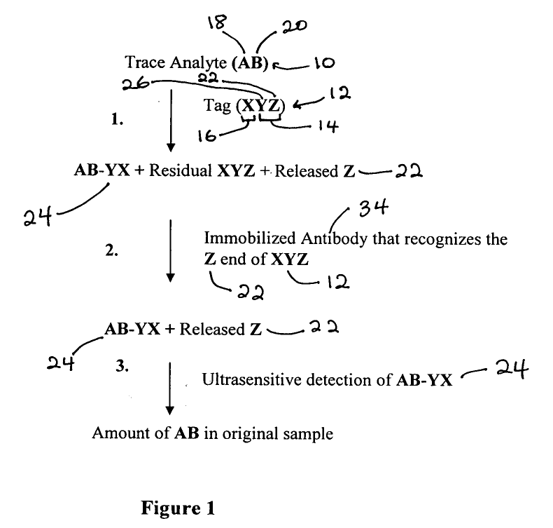

As seen in FIG. 1, trace analyte AB 10 first is derivatized in step 1 with a tag XYZ 12 having a covalently reactive YZ part 14 and nonreactive signal X part 16. A 18 is the part of AB 10 that does not react with XYZ 12, while B 20 is that part that does. Z 22 is the part of XYZ that is displaced in the tagging reaction. The reaction of step 1 yields a derivatized analyte AB-YX 24 in which the Y 26 part of YZ 14 is covalently attached to the B 20 part of analyte AB 10. In addition to general contaminants that are not shown, residual, unreacted XYZ 12 remains in the reaction mixture along with released Z 22. In step 2, the reaction mixture is subjected to affinity chromatography employing an immobilized antibody 34, or other affinity reagent, that recognizes the Z 22 end of XYZ 12. This antibody does not recognize AB-YX 24 because Z is missing in this latter species. Also, via steric hindrance, AB 10 can block any partial recognition the antibody may have for Y 26. This step yields purified AB-YX 24. Released Z 22 will persist whenever it is too small for sufficient recognition by an antibody or other affinity reagent to accomplish its removal. Since released Z 22 has no signal properties, it does not interfere in the subsequent detection step. AB-YX 24 is detected with high sensitivity in step 3 since the excess amount of tag XYZ 12 has been removed, and AB-YX 24 possesses the signal group X 16 that provides high signal strength. Thus, with little tagging noise remaining, the amount of trace analyte AB in the original sample is determined with high sensitivity.

An affinity antibody for use in the method of the invention can be prepared by using albumin-Z 22 or albumin-YZ 14 as an immunogen, where Z 22 or YZ 14 is functioning as a hapten. Z 22 or YZ 14′ conjugated as a hapten to another protein such as key limpet hemocyanin also can be the immunogen.

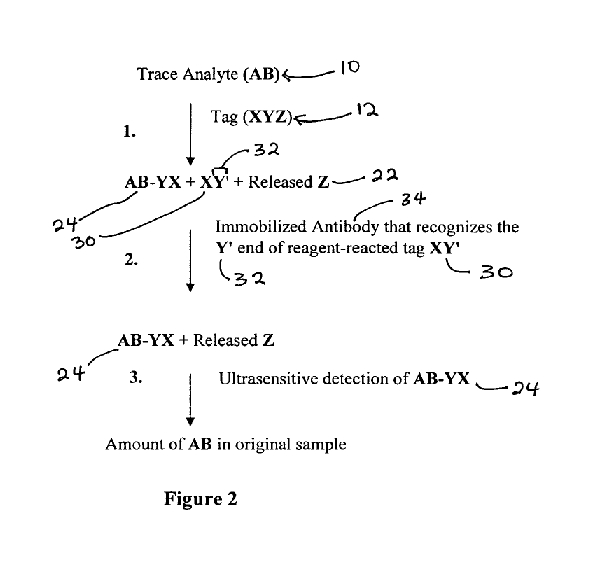

The analytical scheme shown in FIG. 2 is similar to that in FIG. 1, except that in this case a tagging reagent XYZ 12 that is relatively unstable under the conditions of the reaction is employed. This tagging reagent undergoes side reactions with one or more of the reagents (including solvent) in the reaction mixture in parallel with its reaction with trace analyte AB 10. XY′ 30 represents the reagent-reacted (reagent-derived) byproducts of XYZ 12. Ordinarily, the reaction conditions are selected so that just one major XY′ 30 byproduct needs to be removed. In this case, one affinity substance would be adequate. In step 2, the reaction mixture from step 1 is subjected to affinity chromatography purification in which at least one affinity substance 34 is employed that is directed at the Y′ 32 end of at least one XY′ 30 byproduct. This yields purified AB-YX 24, now devoid of significant tagging noise, so that AB-YX 24 can be detected with high sensitivity based on its high signal strength.

This strategy of employing an affinity reagent late in the detection process that is directed towards the reactive part or byproduct thereof of the tag not only greatly reduces tagging noise, but accomplishes this in a practical way. This is because the same affinity reagent(s) can be applied to many different analytes, reducing cost. It has always been a disadvantage that a different affinity reagent is needed for every analyte when analyte-directed affinity is used.

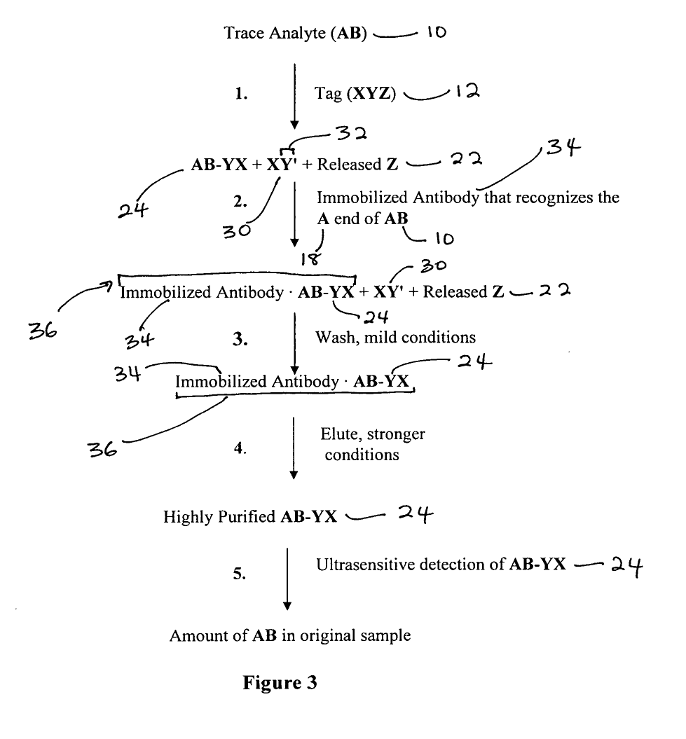

In FIG. 3, a scheme is shown that is similar to that in FIG. 2 except that an immobilized antibody 34 is employed in step 2 that recognizes the nonreactive A 18 end of the analyte AB 10. This yields an affinity complex 36 of the immobilized antibody with the tagged analyte AB-YX 24 along with uncomplexed XY′ 30 and released Z 22. Washing the immobilized antibody under mild conditions removes XY′ 30 and released Z 22 in step 3, while retaining AB-YX. Washing conditions such as water or aqueous ammonium acetate, in which the affinity complex is stable, are good for this purpose. Subsequently, a solvent such as aqueous methanol, methanol, dilute volatile acid, or dilute volatile base is added as an eluting solvent, which breaks up the affinity complex, yielding released, high purified AB-YX 24 in step 4. AB-YX 24 is detected with high sensitivity in step 5.

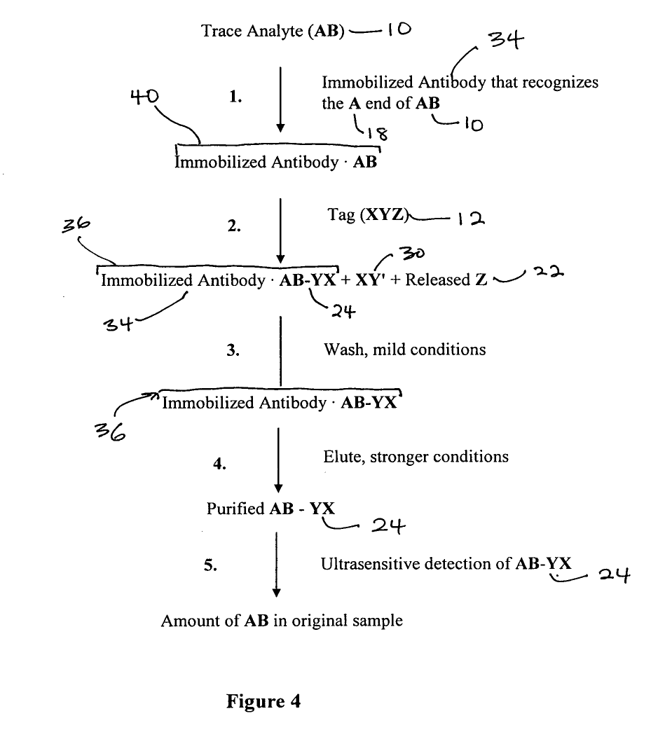

In FIG. 4, trace analyte AB 10 is detected by a scheme in which an affinity substance is introduced before the tagging reaction starts and then continues to complex its target during and after the tagging reaction. In step 1, an immobilized antibody is used as the affinity substance that has been raised against the A 18 end of AB 10 so that it complexes AB in this way, leaving the B 20 end exposed. The tag XYZ 12 in step 2 then reacts with the B 20 part of the complexed-AB 40, yielding a complex 36 of the antibody with AB-YX. Byproducts XY′ 30 and released Z 22 are also present in the reaction mixture after step 2. Washing the immobilized antibody under mild conditions in step 3, as above, in which the antibody•AB-YX complex 36 remains intact, removes these latter chemicals. Affinity captured AB-YX 36 is then eluted in step 4 under stronger washing conditions, such as those cited above. This yields a highly purified AB-YX 24 devoid of tagging noise since XY′ 30 and released Z been removed. Thus, AB-YX is detected with high sensitivity.

For further purification, one or more additional affinity substances can be employed after the tagging reaction directed at the Z 22 end of XYZ 12, or at the Y′ 32 end of XY′ 30, along with an affinity substance directed at the nonreactive part 18 (A) of the analyte.

The affinity step(s) can be applied in various ways in the method. In addition to the technique of affinity chromatography, the well-known techniques of affinity filtration, affinity centrifugation, affinity electrophoresis, and magnetic affinity extraction (paramagnetic particles coated with an affinity reagent are harvested with a magnet) can be used to give the separation of affinity-bound and unbound species.

Use of an affinity substance after a tagging reaction with a recognition property or specificity as described is advantageous in trace analysis via tagging in two major ways. First, it allows tagging-noise to be removed just prior to detection, where it counts the most. When an affinity substance is used only prior to the tagging reaction, contaminants that enter the sample in the later steps of the method are not subjected to removal by an affinity substance, making them more likely to persist and thereby contribute as noise at the detection step. Second, the post-tagging use of an affinity substance provides a convenient way to concentrate the tagged analyte just prior to detection. Usually an affinity reagent is employed in a concentrated form (e.g., a small bed of affinity chromatography particles or small volume of a particulate affinity reagent after centrifugation) so that tagged analyte can be eluted in a small volume. This makes it easier to detect all of the tagged analyte at once, maximizing its signal strength.

General examples of well-known signal substances X that can be used in the invention are as follows: charged organic group, fluorophore (fluorescent group), lumiphore, electrophore, electrochemically-active group, and radionucleotide. An electrophore is a molecular group or compound having a high electron affinity in the gas phase so it can be detected with high sensitivity by an electron capture technique such as gas chromatography with electron capture detection or gas chromatography electron capture mass spectrometry. An electrophore often contains a polyfluoroaryl or polyfluoroalkyl group.

Charged organic signal groups are preferred as signal groups, since such groups can give strong signals in detection by the more common forms of mass spectrometry. General examples of charged organic groups are as follows: quaternary amines, protonated tertiary amines, diprotonated secondary amines, triprotonated primary amines, imidazoliums, pyridiniums, phosphoniums, arsoniums, guanidiniums, thiazoliums, sulfonates, sulfates, sulfoniums and arsonates. Specific examples of charged organic groups are trimethylphenylammonium, triethylphenylammonium, ethyldimethylphenylammonium, N-methylpyridinium, 1-ethylthiazolium, phenylsulfonate, phenylsulfate, and alkylsulfonate. Most preferred are signal groups having a permanent charge.

Examples of detection techniques that can be used, depending on the choice of the signal group X, are as follows: mass spectrometry, luminescence, fluorescence, electrochemical detection and radioactivity counting. Detection by mass spectrometry is preferred, especially with use of charged or electrophore tagging reagents.

Examples of reactive groups YZ that can be used in the invention are as follows: silyl halides, sulfonyl halides, acid halides, acid azides, acid anhydrides, mixed anhydrides, N-hydroxysuccinimides, α-halo ketones, diones, maleimides, diazonium salts, active esters such as imidoesters, aldehydes, halogenated nitrophenyls, arylazides, isothiocyanates, epoxides, carbenes, nitrenes, amines, hydrazides, phenols, hydroxyls, oxyamines, sulfhydryls, and imidazoles.

Examples of affinity substances that can be used in the invention are as follows: peptides, antibodies, antibody fragments, aptamers, receptors, and molecular-imprinted polymers.

Trace analytes for detection by this invention may come from the following classes of analytes: hormones including steroid hormones, neurotransmitters, DNA adducts, carbohydrates, vitamins, lipids, amino acids, peptides, proteins, drugs, metabolites, drug metabolites, combinatorial synthesis products, toxins and environmental contaminants such as pesticides. Specific examples of trace analytes are estradiol, testosterone, and 1,25-dihydroxyvitamin D.

The following examples are presented to illustrate the advantages of the present invention and to assist one of ordinary skill in making and using the same. These examples are not intended in any way otherwise to limit the scope of the disclosure.

### EXPERIMENTAL

3-Sulfopropionic acid N-hydroxysuccimide ester (SPA-NHS) and 4-sulfobenzoic acid N-hydroxysuccinimide ester are (SBA-NHS) are known compounds (Keough, T., Youngquist, R. S., Lacey, M. P., 2003. Sulfonic Acid Derivatives for Peptide Sequencing by MALDI MS, Anal. Chem. 75, 156A-165A). Reaction of SPA-NHS with histamine yields sulfopropylhistamine (SPH). Reaction of SBA-NHS with histamine yields sulfobenzoylhistamine (SBH). 6-(trimethylamino)hexanoic acid N-hydroxysuccinimide ester (TMAHA-NHS) is prepared as described (Bartlet-Jones, M., Jeffrey, W. A., Hansen, H. F., Pappin, D. J. C., 1994. Peptide ladder sequencing by mass spectrometry using a novel, volatile degradation reagent, Rapid Commun. Mass Spectrom. 8, 737-742). N-Acetyltyrosine sulfate-N-hydroxysuccinimide carboxyl ester (NATS-NHS) is prepared by reacting N-acetyltyrosine sulfate with N-hydroxysuccinimide in the presence of N-(3-dimethylaminopropyl)-N′-ethylcarbodiimide.

### EXAMPLES

### Example 1

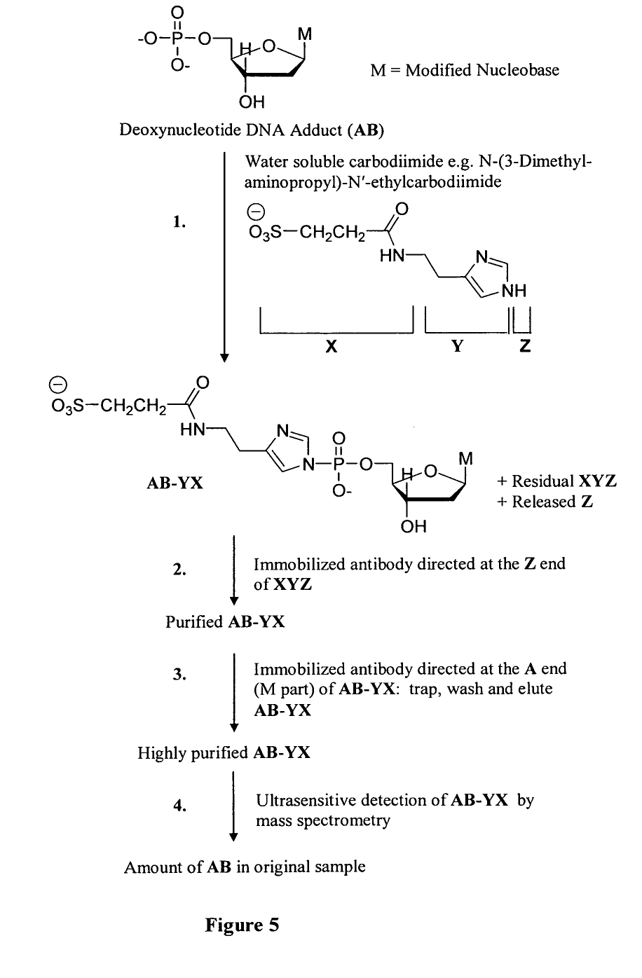

This example, which represents the scheme according to FIG. 1 with an additional purification step, is illustrated in FIG. 5. A DNA adduct (analyte AB) in a nucleotide form in water can be detected by covalent labeling of its phosphate group in step 1 with SPH, a molecular tag XYZ comprising a sulfopropyl signal group X at one end and an imidazole reactive group YZ at the other end. Residual tag XYZ is removed in step 2 on an affinity chromatography column using an immobilized anti-imidazole antibody 34 directed at the Z end of XYZ, where the imidazole reactive group is located. Purification of the tag-DNA adduct conjugate 24 is carried out in step 3 on an immobilized anti-A antibody affinity chromatography column prior to detection in step 4 by mass spectrometry. The antibody in step 2 does not recognize AB-YX because AB-YX does not contain an unsubstituted, terminal imidazole group, which is required as the dominant part of the ligand (binding partner) for the antibody used in step 2.

### Example 2

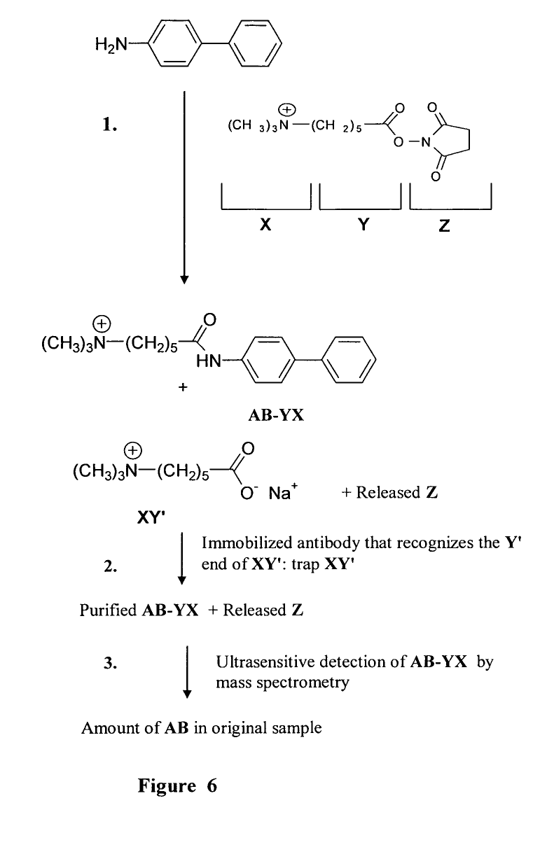

This Example, an illustration of the reaction scheme according to FIG. 2, is shown in FIG. 6. Detection of 4-aminobiphenyl analyte AB is started by covalent labeling of the analyte in step 1 with TMAHA-NHS, a molecular tag XYZ comprising a trimethylamino signal group X at one end and a hexanoic acid N-hydroxysuccinimide ester reactive group YZ at the other end. The product mixture is subjected to affinity chromatography on a column containing an immobilized antibody 34 that recognizes the alkylcarboxylate moiety XY′ (formed by hydrolysis of the N-hydroxysuccinimide ester tag XYZ). The tagged 4-aminobiphenyl conjugate 24, upon elution from the column, is detected in step 3 by liquid chromatography electrospray ionization mass spectrometry. Released Z (N-hydroxysuccinimide) does not contribute noise to the detection step since it has no signal group.

### Example 3

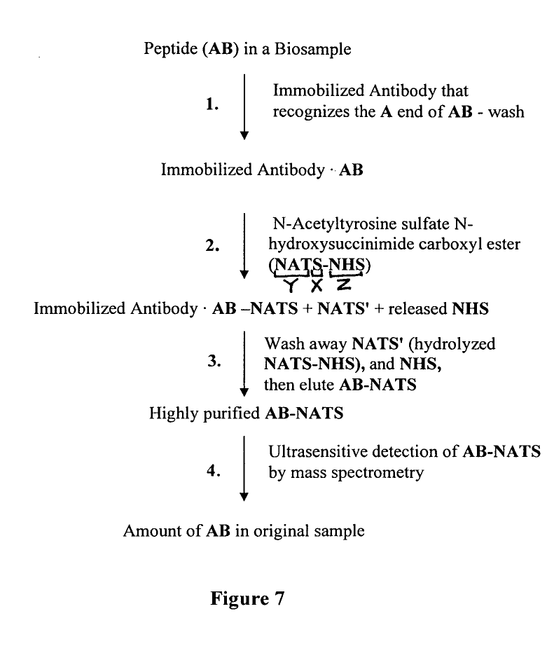

This Example, illustrated in FIG. 7, represents the scheme shown in FIG. 4. A peptide AB is detected by first isolating the peptide in step 1 on an affinity column containing an immobilized antibody 34 that recognizes the peptide in a way that leaves a primary amine moiety of the peptide exposed. While still attached to the antibody, the peptide is labeled in step 2 with N-acetyltyrosine sulfate N-hydroxysuccinimide carboxyl ester (NATS-NHS) as molecular tag XYZ. After nonanalyte species are washed away with water, the N-acetyltyrosine sulfate peptide conjugate 24 is eluted from the affinity column in step 3 with an organic or aqueous/organic solvent, and the conjugate is detected in step 4 by mass spectrometry. AB-NATS also can be eluted from the antibody prior to step 2, and then captured on a fresh portion of this antibody prior to step 3.

### Example 4

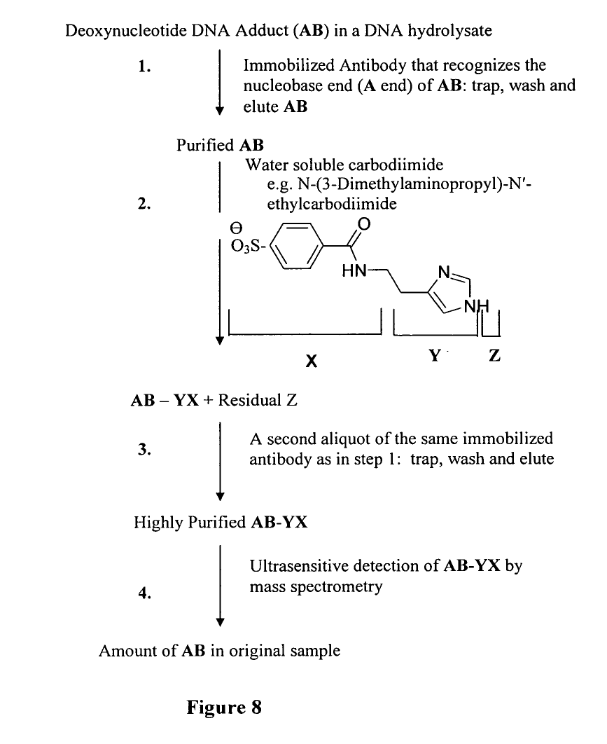

This Example is illustrated in FIG. 8. A DNA adduct AB is detected by first extracting it in step 1 on an affinity chromatography column containing an immobilized antibody 34 that recognizes the nonphosphate (nonreactive) part of a DNA adduct. (Construction of this affinity reagent is described in Morinello, E. J., Ham. A-J. L., Ranasinghe, A., Sangaiah, R., Swenberg, J. A., 2001. Simultaneous Quantitation of N²,3-Ethenoguanine and 1,N²-Ethenoguanine with an Immunoaffinity/Gas Chromatography/High-Resolution Mass Spectrometry Assay, Chem. Res. Toxicol., 14, 327-334.) After nonanalyte substances are washed away, the adduct is eluted and labeled on its phosphate group in step 2 with molecular tag SBH in the presence of a water-soluble carbodiimide. The resulting SBH-DNA adduct conjugate 34 is purified in step 3 using the same affinity reagent as above and the conjugate then is detected in step 4 by mass spectrometry. This example represents the scheme shown in FIG. 3 with the addition of a preliminary purification step.

### Example 5

In another example of the scheme shown in FIG. 3, estradiol is detected by first labeling its 3-hydroxy group with tagging reagent dansyl chloride as described (Xia, Y-Q., Chang, S. W., Patel, S., Bakhtiar, R., Karanam, B., Evans, D., 2004. Trace level quantification of deuterated 17 β-estradiol and estrone in ovariectomized mouse plasma and brain using liquid chromatography/tandem mass spectrometry following dansylation reaction, Rapid Commun. Mass Spectrom, 18, 1621-1628). The dansyl-estradiol complex next is purified on an antibody affinity chromatography column in which the immobilized antibody recognizes the 17-hydroxy region of the dansyl-estradiol. After washing and release, the dansyl-estradiol conjugate is detected by mass spectrometry.

### USE

The assay according to the invention is especially useful for detection of trace analytes in clinical samples by mass spectrometry. Currently, many of these analytes, such as estrogens and peptide hormones, are detected by immunoassays, giving rise to the clinical immunodiagnostic market which is valued at $6 billion per year. There are problems with accuracy, precision, sensitivity and cost for these immunoassays. These problems can be substantially overcome through the use of mass spectrometry due to its advantages of utilizing stable isotope internal standards, providing measurements of analytes with high resolution and measuring multiple analytes simultaneously. However, many of the clinical analytes need to be tagged for ultrasensitive detection by mass spectrometry. Unfortunately, current techniques for overcoming tagging noise are not effective or practical for analysis of real samples. This is especially important for charge tags since tagging noise from charge tags tends to be persistent and intense. By overcoming the problem of tagging noise in the disclosed practical way, the assay according to the invention promises to improve health care by replacing immunoassays with mass spectrometry for the measurement of trace clinical analytes.

While the present invention has been described in conjunction with a preferred embodiment, one of ordinary skill, after reading the foregoing specification, will be able to effect various changes, substitutions of equivalents, and other alterations to the compositions and methods set forth herein. It is therefore intended that the protection granted by Letters Patent hereon be limited only by the definitions contained in the appended claims and equivalents thereof.

What is claimed is:

1. In an assay for detecting the amount of a trace analyte AB in a sample, the assay comprising the steps of:
providing an assay mixture comprising a sample in which an amount of a trace analyte AB is to be determined;
contacting the analyte AB in the assay mixture with a tagging reagent XYZ, to give the covalent product AB-YX along with residual XYZ and/or byproducts XY′, wherein B is the part of analyte AB that reacts with XYZ, A is the nonreactive part of AB, X is a signal group in XYZ, YZ is a reactivity group in XYZ, Z is the part of the reactivity group YZ that is replaced by AB, and XY′ is any product that forms when XYZ reacts with a reagent including solvent;
and measuring the amount of AB-YX in a free form from the assay mixture, wherein the amount of AB-YX from the assay mixture gives the amount of trace analyte AB in the sample;
the improvement comprising, contacting the assay mixture with at least one affinity substance at least after the step of contacting the analyte AB in the assay mixture with a tagging reagent XYZ, wherein said at least one affinity substance is directed at the YZ part of XYZ, the Y′ part of XY′, or the A part of AB-YX.

2. The assay of claim 1, wherein the assay mixture is contacted with the affinity substance before contacting the analyte AB in the assay mixture with the tagging reagent XYZ and wherein the affinity substance remains in the assay mixture during the step of contacting the analyte AB in the assay mixture with the tagging reagent XYZ.

3. The assay of claim 1, wherein AB-XY is detected by fluorescence or by mass spectrometry.

4. The assay of claim 1, wherein at least one affinity substance is a solid phase reagent.

5. The assay of claim 1, wherein at least one affinity substance is selected from the group consisting of immobilized or free forms of peptides or proteins.

6. The assay of claim 1, wherein at least one affinity substance is selected from the group consisting of immobilized and free forms of peptides, antibodies, antibody fragments, aptmers and molecular-imprinted polymers.

7. The assay of claim 1, wherein X comprises a charged organic group or an electrophoric group.

8. The assay of claim 1, wherein X comprises a fluorophore, lumiphore, electrochemically-active group, or radionucleotide.

9. The assay of claim 1, wherein AB is a steroid, lipid, carbohydrate, peptide, vitamin, metabolite drug, drug metabolite or DNA adduct.

10. The assay of claim 1, wherein a reactive part (B) of analyte AB is labeled with tag XYZ forming AB-YX, AB-YX is extracted by an affinity substance directed towards its A part, and AB-YX is detected by fluorescence or mass spectrometry.

11. The assay of claim 10, wherein AB is complexed with the affinity substance during its reaction with XYZ.

12. The assay of claim 10, wherein AB is a steroid, lipid, carbohydrate or vitamin.

13. The assay of claim 10, wherein AB is a metabolite, drug, drug metabolite, peptide or DNA adduct.

14. The assay of claim 10, wherein AB is a protein.

15. The assay of claim 10, wherein the affinity substance is an immobilized form of a peptide or protein.

16. The assay of claim 10, wherein the affinity substance is an antibody or antibody fragment.

17. The assay of claim 10, wherein X comprises a charged organic group or an electrophoric group.

18. The assay of claim 1, wherein a reactive part B of analyte AB is covalently labeled with tag XYZ giving AB-YX along with residual XYZ and/or reagent-derived byproducts XY′, XYZ is extracted with an affinity substance directed towards its YZ part and/or at least one XY′ is extracted with an affinity substance directed at its Y′ part, AB-YX is extracted with an affinity substance directed at its X part or A part, and AB-YX is detected.
